(19) United States
(12) Patent Application Publication
(10) Pub. No.: US 2015/0018645 A1
(43) Pub. Date: Jan. 15, 2015

(54) DISPOSABLE CALIBRATION END-CAP FOR USE IN A DERMOSCOPE AND OTHER OPTICAL INSTRUMENTS

(71) Applicants: Daniel Farkas, Beverly Hills, CA (US); Nicholas MacKinnon, Vancouver (CA); Fartash Vasefi, Beverly Hills, CA (US)

(72) Inventors: Daniel Farkas, Beverly Hills, CA (US); Nicholas MacKinnon, Vancouver (CA); Fartash Vasefi, Beverly Hills, CA (US)

(21) Appl. No.: 14/332,254

(22) Filed: Jul. 15, 2014

Related U.S. Application Data
(60) Provisional application No. 61/846,525, filed on Jul. 15, 2013.

(51) Int. Cl. A61B 5/00; A61B 19/00

(52) U.S. Cl. CPC A61B 5/0077; A61B 5/441; A61B 5/0075; A61B 5/0071; A61B 19/44; A61B 5/0082; A61B 2019/442; A61B 2019/448; A61B 2019/446
USPC 600/317; 600/476; 600/477

(57) ABSTRACT

A disposable tubular end cap for a dermoscope for examining the skin of an animal. The end cap has one end for receiving light from and transmitting light to the dermoscope, a removable calibration target disposed at the other end having optical characteristics similar to a standard skin type, and an identifier disposed on the end cap. The identifier uniquely identifies the end cap and associates it with both data regarding light emitted from the skin of an animal and calibration data derived from the calibration target. Animal tissue may be examined to identify lesions using a plurality of wavelengths and a plurality of polarizations.

### REFERENCE TO RELATED APPLICATION

Applicants hereby claim the benefit of priority under 35 U.S.C. §120 to Farkas et al. U.S. Provisional Patent Application No. 61/759,910, filed Feb. 1, 2013 and entitled METHOD AND SYSTEM FOR CHARACTERIZING TISSUE IN THREE DIMENSIONS USING MULTIMODE OPTICAL MEASUREMENTS, the contents of which are hereby incorporated by reference in this disclosure.

### FIELD OF THE INVENTION

The present invention relates to accessories for optical human or animal tissue examination instruments, particularly to a coupling and calibration accessory that can be attached to a hand-held optical probe for tissue live examination and pathology. Hereafter, wherever the term “animal” is used in the specification, claims, drawings or abstract, it is intended to and shall encompass non-human animals in the general scientific sense as well as human beings.

### BACKGROUND OF THE INVENTION

Dermoscopy is the examination of skin with an instrument known as a dermoscope to identify skin lesions or other pathology. It is a widely accepted tool for examining the abnormalities of skin conditions including pigmented lesions, especially helpful in diagnostics of various skin cancer conditions. Giuseppe Argenziano et al, “Dermoscopy Improves Accuracy of Primary Care Physicians to Triage Lesions Suggestive of Skin Cancer,” J Clin Oncol 2006; 24:1877-1882. A traditional dermoscope has several components, including a magnifier, a light source that is nonpolarized, a transparent plate and a liquid coupling medium between the dermoscope and the skin. The typical strength of the magnifier is 3×-10×. The dermoscope allows a clinician to observe and analyze skin lesions without the obstruction of skin surface reflections.

Modern dermoscopy has advanced beyond mere magnification to the use of digital imaging techniques that employ multiple illumination wavelengths and polarization orientations, to examine the skin's subsurface features that are not observable merely with the naked eye. Darrell S. Rigel, M D et al. “The Evolution of Melanoma Diagnosis: 25 Years Beyond the ABCDs,” Ca Cancer J Clin 2010; 60:301-316. With the advent of multi-wavelength (sometimes called multispectral) and hyperspectral imaging techniques, more complex dermoscopes have been introduced to capture the skin image formed by diffused light from the illuminated skin at different wavelengths and polarizations.

Skin is generally classified as belonging to certain skin types which are dependent on the kind of melanin present in the tissue and its concentration S. Del Bino, et al., “Relationship between skin response to ultraviolet exposure and skin color type,” 2006 Pigment Cell Res. 19; 606-614. Areas of skin from different body sites also provide a wide range of optical properties (such as absorption and fluorescence) due to their physiological and anatomical characteristics (such as the amounts of melanin, collagen, blood and other components) even within a specific skin type. Different anatomical sites have different conformations which can affect the ability of a measurement device to easily access them. For example, the area next to the nose, ear, or eye is more difficult to access than a broad flat area of the back. Dermoscopes used to examine these different areas of skin need to conform to the site being measured to facilitate accurate observations with acceptable stability. Acceptable stability requires minimal axial, lateral, rotational and angular movement of the dermoscope during the scanning procedure.

Calibration targets are commonly used in reflection-based multispectral and hyperspectral imaging systems to extract the instrument spectral response, including the effect of the light source, detector spectral sensitivity and light transmission properties of system optics. The calibration targets also help to identify spatial variations due to illumination source, transmission optics, or detector characteristics. To maintain high measurement accuracy in multispectral and hyperspectral advanced dermoscopy, the system should be calibrated before the skin examination. However, the varying optical characteristics and unique skin surface of each individual present a calibration challenge in modern multispectral and hyperspectral dermoscopy.

In addition, it is vital that a dermoscope which is used for examination of multiple patients provides a way of ensuring that it is aseptic, i.e. there is no transmission of even trace amounts of any contamination, such as potentially infectious agents, between patients.

### SUMMARY OF THE INVENTION

The accessory device is a disposable end-cap with a unique identification, a removable calibration target that has responses graduated for various skin types as well as conformations suitable for various anatomical locations.

An end cap for use with a dermoscope having a scope aperture adapted to emit light to illuminate animal tissue and receive light emitted from the tissue in response to illumination of the tissue, and a data processor adapted to process data regarding light emitted from the animal tissue is disclosed. The end cap comprises, a tube having a first end forming a tube aperture adapted to receive light from and transmit light into the scope aperture, and a second end; a calibration target adapted to be removably disposed at the second end of the tube so as to receive light from the dermoscope through the tube aperture and the scope aperture; and an end cap identifier disposed on the end cap so as to uniquely identify the end cap so that the data processor may associate the end cap with data regarding light emitted from tissue of an individual subject and calibration data derived from light received from the calibration target.

A method for calibrating a dermoscope having a scope aperture adapted to emit light to illuminate the animal tissue and receive light emitted from the tissue in response to illumination of the tissue, and a data processor adapted to process data regarding light emitted from the animal tissue is disclosed. The method for calibrating comprises: identifying the skin type of an subject whose skin is to be examined; selecting an end cap corresponding to the skin type of the subject, the end cap having a tube having a first end forming a tube aperture adapted to receive light from and transmit light into the scope aperture, and a second end, and a calibration target adapted to be removably disposed at the end of the tube so as to receive light from the dermoscope through the tube aperture and the scope aperture, the calibration target corresponding to the selected skin type; entering data regarding the optical characteristics of the calibration target into the data processor; and causing the data processor to calibrate the response of the dermoscope to take into account the assumed optical characteristics of the skin based on the optical characteristics of the calibration target.

A method for examining animal tissue to identify lesions is also disclosed. This method comprises providing a dermoscope having a scope aperture adapted to emit light to illuminate a portion of the tissue and receive light emitted from the tissue in response to illumination of the tissue, and a data processor adapted to process data regarding light emitted from the tissue; providing an end cap adapted to be placed on the scope aperture, the end cap having a calibration target and a unique identifier representing the type of end cap and optical characteristics of the calibration target; reading the unique identifier to provide to the data processor the type of end cap and optical characteristics of the calibration target; placing the end cap on the scope aperture; taking one or more measurements of the calibration target; calibrating the dermoscope based on the measurements of the calibration target, the type of calibration target and the optical characteristics of the calibration target; removing the calibration target from the end cap; illuminating the tissue over a plurality of wavelengths and a plurality of polarizations while measuring the reflected illumination light; and based on the preceding measurements of intensity, producing data representative of one or more characteristics of the tissue.

It is to be understood that this summary is provided as a means for generally determining what follows in the drawings and detailed description, and is not intended to limit the scope of the invention. The foregoing and other objects, features, and advantages of the invention will be readily understood upon consideration of the following detailed description taken in conjunction with the accompanying drawings.

### BRIEF DESCRIPTION OF THE DRAWINGS

FIG. 1 is a side view of a representative hand-held probe of a dermoscope, to which an end-cap according to the invention, shown in phantom, may be attached.

FIG. 2 is a perspective of a work station of the representative dermoscope of FIG. 1.

FIG. 3 is a schematic representation of a first embodiment of a disposable end-cap according to the invention for a dermoscope probe, adapted to access a relatively large anatomical field.

FIG. 4A is a perspective of a representative calibration target for skin types I and II.

FIG. 4B is a perspective of a representative calibration target for skin types III and IV.

FIG. 4C is a perspective of a representative calibration target for skin types V and VI.

FIG. 5A is a perspective of a first alternative calibration target configuration where identification information is recorded within the visible aperture of the dermoscope.

FIG. 5B is a perspective of a second alternative calibration target configuration where identification information is recorded within the visible aperture of the dermoscope.

FIG. 6 is a schematic representation of a second embodiment of a disposable end-cap according to the invention for a dermoscope probe, adapted to access a relatively smaller anatomical field.

FIG. 7 is a block diagram of a multimode dermoscope of a type with which an end-cap according to the invention may be used, showing the end-cap configured for calibration.

FIG. 8 is a flowchart of a method for setting up a multimode imaging dermoscope for calibration and skin measurement using an end-cap according to the invention.

FIG. 9 is a flowchart of a calibration method for a multimode imaging dermoscope using an end-cap according to the invention.

FIG. 10 is a block diagram of a multimode dermoscope of a type with which an end-cap according to the invention may be used, showing the end-cap configured for skin examination.

FIG. 11 is a flowchart of a method for examining skin with a multimode dermoscope and an end-cap according to the invention.

### DETAILED DESCRIPTION OF EMBODIMENTS OF THE INVENTION

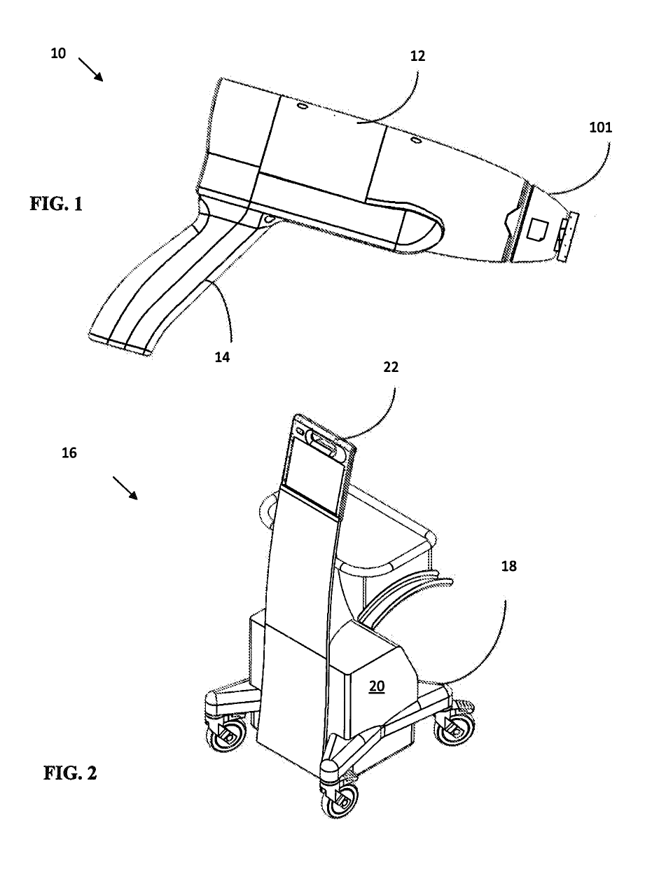

Referring to FIGS. 1 and 2, a modern optical multimode dermoscope for tissue examination and for pathology of the type with which an embodiment of the present invention most likely would be used would ordinarily include a hand-held probe 10 having a light transmitter and receiver 12 supported by a handle 14, and a data processor workstation 16 supported, for example, by a cart 18 and including a programmed computer within a console 20 with an input and output device such as a touchscreen display 22. A multimode light source is also disposed within the console. While various modes of optical examination and data processing might be employed, a preferred approach to dermoscopy with which the end cap of the present invention may be used is disclosed in the '910 application.

In general, modern multispectral and hyperspectral dermoscopes collect complex optical data in order to function as medical diagnostic devices. Such instruments need to be calibrated before their use to cancel out variations caused by the instrument performance such as light source variations (e.g., an aging light source) or changing environmental conditions (e.g., room temperature). The instrument should be calibrated with respect to its response as a function of wavelength and polarization of light. The instrument should also be calibrated to take into account any spatial variations across the field of view caused by the instrument, (e.g., brightness variation due to the illumination). After calibration, the data measured will only be dependent on the tissue optical properties.

The accuracy, reliability and safety of such modern dermoscopes can be improved when calibrated with a single-use disposable end-cap that includes a calibration target having optical properties that are optimized according to the patient skin type. The embodiments of the subject end-cap described herein can be used in a variety of multispectral and hyperspectral dermoscopes and are not limited to use with the preferred multimode optical imaging system disclosed in the '910 application.

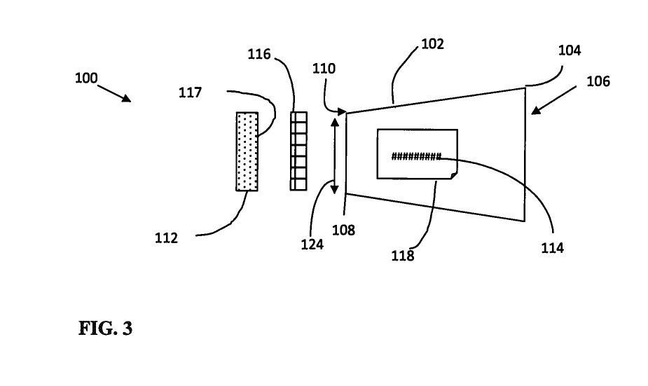

Referring more specifically to FIG. 3, one preferred embodiment of the present invention comprises an end cap 100 for a multimode optical dermoscope, such as that disclosed in the '910 application. The end cap 100 enables calibration of the dermoscope based on skin type and anatomical location of the skin examination, provides protection for the dermoscope optics, and provides an aseptic barrier from the skin. More specifically, end cap 100 comprises a tube 102, having a first end 104 including a first aperture 106 and a second end 108 including a second aperture 110, a removable calibration target 112, and a unique identifier 114. A similar alternative end cap 101 having a frustro-convex tube is shown attached, for example, to the probe 10 in FIG. 1. It is to be understood that various shapes of the end cap may be used, depending on the anatomical location of the skin to be examined and other relevant considerations.

An elastomeric ring 116 may be provided between end 108 of the tube 102 and the calibration target 112. When the target is removed and the ring 116 is placed against tissue to be examined, the ring both cushions the skin and provides a light-tight seal against the variable skin surface, which, together with the tube 102, acts as a barrier preventing ambient light from entering the illumination and detection paths of the dermoscope. The ring also provides partial compensation for the effect of operator pressure which may deform the examined skin and may reduce slippage of the end cap when placed against the skin, which could otherwise cause image blur or mis-registration. The tube itself can also be shaped to optimize stability and accessibility for skin examination at different anatomical locations.

The removable calibration target 112 is preferably provided with a releasable adhesive 117. The adhesive enables the target to be attached to the second end 108 of the tube, where no elastomeric ring is provided, or to the elastomeric ring 116 when it is provided. Once the dermoscope is calibrated, as discussed below, the calibration target may be removed by pulling it away from the tube, or the ring, so as to break the grip of the adhesive.

The calibration target is mounted on the end-cap with its inner face disposed at approximately the plane where the tissue will be during an examination. Ordinarily, the calibration target will remain in place during the system calibration procedure but will be removed prior to the patient examination. Removal of the calibration target from the end-cap will present an aseptic surface for contact with the patient skin. The purpose of the aseptic surface is to provide a clean sanitary surface for each new patient to assist with infection control and to prevent the transmission of communicable diseases.

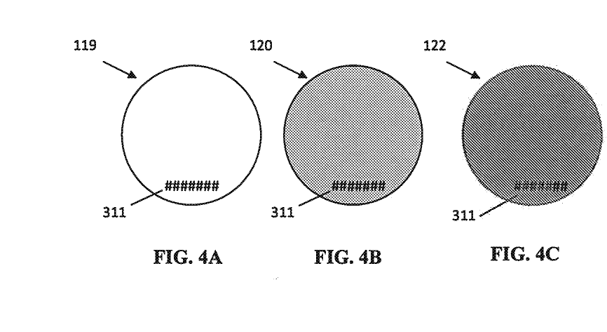

It is important for the optical properties of the calibration target to be optimized according to the patient skin type to improve system accuracy as a result of calibration. In dermatology, clinical practitioners classify skin by its appearance into a number of skin types. “Relationship between skin response to ultraviolet exposure and skin color type”, S. Del Bino et al. Pigment Cell Res. 19; 606-614 (2006) Currently there are six commonly accepted skin types from skin type I (white pale skin) to skin type VI (black skin). Each of these skin types can reemit a specific portion of illuminated light due to their specific absorption and scattering properties. To provide the best accuracy for skin examination, the system should be calibrated for the range of remitted light for that skin type. To that end, a number of different types of calibration targets should be provided to obtain the best accuracy and dynamic range for system calibration and skin examination procedures. Preferably at least two calibration target types should be provided and as many as six may be advantageous, but for most purposes it is believed that three calibration targets, namely one target 119 shown by FIG. 4A for skin types I and II, another target 120 shown by FIG. 4B for skin types III and IV, and a third target 122 shown by FIG. 4C for skin types V and VI, should be provided. The tube 102 and the calibration target 112 can be color coded to facilitate selection of the appropriate end-cap for a specific skin type and anatomical location. The calibration target should be referenced to known standards such as those provided by The United States National Institute of Standards and Technology (“NIST”) and with an identifier (e.g. serial number) to ensure the calibration target optical properties match the requirements of the patient's skin to be examined and are traceable to the primary standard. One suitable reference calibration target material that is commercially available is Spectralon®, provided by Labsphere In., North Sutton, N.H. Another example of a suitable reference target material that is commercially available is “polyurethane phantoms’, Biomimic™, provided by INO Inc., Quebec City, Canada.

Referring again to FIG. 3, the identifier 114 may comprises a code having a unique combination of numbers, letters or other symbols, which can be interpreted by the system software to uniquely identify the end cap and associate it with a particular anatomical location for skin examination and with a particular calibration target corresponding to skin type. Alternatively, the identifier may comprise a unique one- or multi-dimensional symbol. Preferably, the identifier is also used to associate the end cap with a particular individual examined, or to be examined, and with a particular dermoscope, or type of dermoscope, with which the end cap is used. The identifier may be visible to the human eye or to a scanner that may operate in a spectrum outside what is visible to the human eye, such infrared wavelengths. The identifier may be printed on the end cap and the calibration target or, for example, on an adhesive tag 118 attached to the end cap.

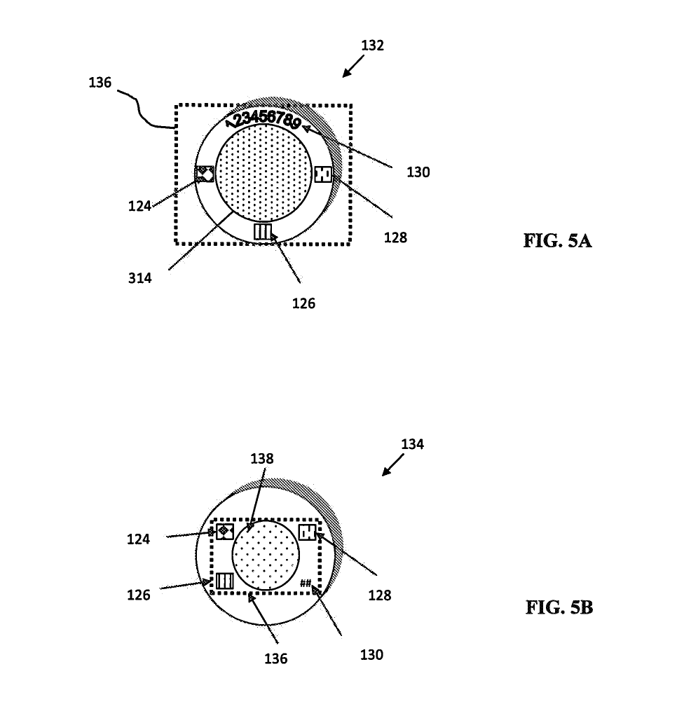

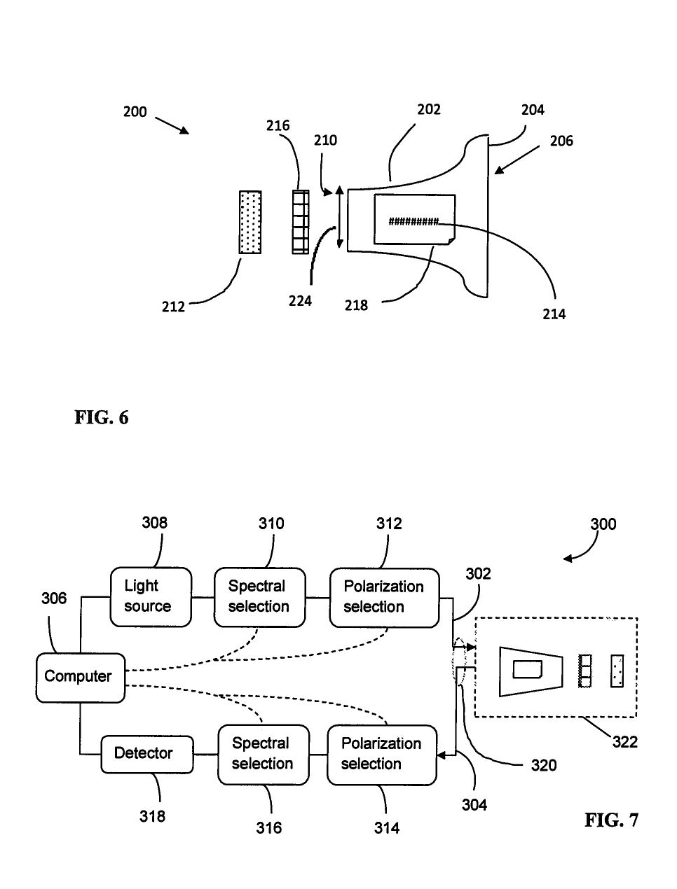

Turning to FIG. 5A, optical features such as barcode or color code identifier 124, spatial resolution bars 126, polarization test target 128, or identification numbers 130 can be incorporated in a calibration target 132. The barcodes can be one dimensional or two-dimensional barcodes. The color-code identifier can be a material with a specific wavelength-dependent response to illumination. The wavelength-dependent response can be, for example, material having a characteristic response applied to the target or a label applied to the target and having an identifier presented therein. The label may comprise color-paint, holograms, nanoparticles, or quantum dots with predesigned spectral properties. The color code can be interpreted by analysis of the images captured from the dermoscope. The polarization test target has specific polarization response due to its material or structure and can be used to test the linear and cross polarization performance of the detection system. The spatial resolution and polarization test targets may be combined with any of the color code identifier, barcode or identification numbers.

Referring to FIG. 6, a third, alternative embodiment of an end cap 200 has a tube 202 that, unlike the frustum-shaped end cap 100 of FIG. 3, or the frusto-conical shaped end cap 101 in FIG. 1, has a frusto-conical-convex-concave shape leading from a wide aperture 206 and a first end 204 for accommodating the dermoscope to a relatively narrow aperture 210 having a diameter 124 that is much less than the diameter 124 of end cap 100, thereby enabling the examination of a much smaller anatomical area than that enabled by end cap 100. Accordingly, the end cap 200 has a calibration target 212 and an optical elastomeric ring 216 that are likewise smaller in diameter. Identifier 214 and the sticker 218 with which it is attached to the tube 202 are different from those of the end cap shown in FIG. 3 in that, among other things, the identifier 214 is associated with a different skin surface area, or anatomical location, than the identifier 114 of FIG. 3.

The calibration target may vary based on the shape and size of the second end of the end cap tube, i.e., circular and large in this case of end cap 100 or circular and small in the case of end cap 200. Referring now to FIG. 5B as well as FIG. 5A a calibration target disk may be sized to encompass the sensor field of view 136 (in the case of target embodiment 132) or the calibration disk may be sized to encompass the sensor field of view 136 (in the case of target embodiment 134). Features on the calibration target such as barcode 124 or color code identifier 123, spatial resolution bars 126, polarization test target 128, and identification numbers 13—may be disposed in the sensor field of view 136 but outside the tissue field of view 138 or they may be distributed at the periphery of the tissue field of view 138.

In use, the identifier may be manually entered into the data processor of the dermoscope by a keyboard, particularly in the case of a code visible to the human eye. The identifier may be a one or 2D barcode that is scanned by a barcode scanner for input to the processor or a multi-dimensional symbol that is captured by an imaging device. Alternatively, the identifier may be represented by an encoded signal produced by an RFID tag that is attached to the end cap and can be read by an RFID scanner. In another embodiment the identifier may be incorporated in the field of view of the imaging device adjacent to the removable calibration target and the tissue field of view.

The unique identifier 114 or 214 provided with each end-cap is used to inform the system software what type of end-cap and what type of calibration target has been placed on the hand-piece. The identifier allows the system software to recognize the calibration target and adjust the image capture parameters for the calibration and patient measurement procedures based on the type of calibration target being used. For example, type IV skin may require a longer image exposure time at certain wavelengths. The system may also adjust other image capture parameters such as camera gain, dynamic range, or pixel binning.

The identifier 114 or 214 also allows the system software to determine if the end-cap has been previously used or if too much time has elapsed since calibration. If the end-cap has been used, it may be unsanitary and should be discarded. A new end-cap should be used and a new calibration should be performed. If too much time has elapsed since the end-cap was used for calibration, it may have been exposed to ambient contamination, may be unsanitary and should be discarded. A new end-cap should be used and a new calibration be performed. If too much time has elapsed since the end-cap was used for calibration, the calibration may no longer be valid due to the system variations and a new calibration be performed.

The identifier 114 or 214 allows the system software to recognize the shape of the end-cap tube and configure the imaging system accordingly. In some embodiments, the end-cap is shaped to provide enhanced access to a small area of the tissue. When the image is captured, the field of view may be reduced compared to the standard imaging field of view. The system software can adjust the size of image it needs to capture, the speed of acquisition, or the system resolution to provide optimal imaging for the specific anatomical region or for the specific end-cap.

Referring again to FIG. 1, the probe 10 is used by an operator to make measurements at different anatomical locations of an animal subject, ordinarily a human being. The probe provides illumination, wavelength and polarization selection, and detection components of the dermoscope. The probe is connected to and controlled by the work station 16, which comprises system controllers, light sources, data acquisition interfaces, operator interfaces, and optional components such as barcode readers or RFID scanners, as well as the system software. The end-cap 100 (shown in phantom) can be removably attached to the probe. When the hand-piece is not in use, a dust-cap can be removably attached to the hand-piece. When the system is used for skin measurements, it must be allowed to warm up and equilibrate after it is turned on. After the system software determines that enough time has passed for the system to stabilize, the dust-cap can be removed and the end-cap can be attached to the probe. The unique identifier of the probe can then be entered or scanned for recognition by the system software.

Multimode optical imaging system software in the workstation uses the unique identifier to identify the characteristics of the end-cap and calibration target being used from a database of known characteristics. It then configures the multimode optical imaging system settings, such as field of view, exposure time for each waveband, and the like. The system software then makes calibration measurements on the calibration target to normalize the system response for the type of the skin being imaged. After the calibration procedure, the calibration target will be removed from the end-cap allowing access to the aseptic surface and skin-imaging procedure can begin.

A representative block diagram of a dermoscope system 300 for capturing and processing multimode optical measurements is shown in FIG. 7. The system comprises an illumination beam path 302 for presenting illumination light to an area of tissue to be examined, an emitted light capture path or other data processing unit 306 for controlling the illumination and detected light and processing the detected light. The illumination beam path 302 comprises a light source 308, an illumination spectral selection unit 310, and an illumination polarization selection unit 312. The emitted light capture path comprises an emitted light polarization selection unit 314, and emitted light spectral selection unit 316, and a detector 318. The illumination light source 308 may be at least one of a broadband lamp, such as tungsten or an arc lamp, a single wavelength laser, a multi-wavelength laser, a super continuum laser, a light emitting diode, or similar sources now or hereafter known in the art. The spectral selection units 310 and 316 may be an optical filter, an optical filter wheel, a diffraction grating, a liquid crystal tunable filter, an acousto-optic tunable filter, a plasmonic-based spectral selection device such as a metallic nanostructure, or similar spectral selection devices now or hereafter known in the art. The polarization selection units 312 and 314 may be conventional polarizers such as rotatable crystal or wire grid polarizers or liquid crystal variable retarders, plasmonic metallic nanostructure based filters, or similar devices now or hereafter known in the art. The optical system 320 may comprise free space optics, such as lenses, mirrors and prisms, fiber optics, integrated optics, liquid light guides, or other technology now or hereafter known in the art that can perform the same function.

The illumination light source 310 may, for example, comprise a Xenon arc lamp incorporated in a spectral programmable light source, such as the product sold under the mark OneLight® Spectra by OneLight Corporation, Vancouver, BC, polarized in only one linear state. The detected light from the tissue sample can be divided into two optical paths comprising cross and parallel polarizations using a beam-splitter and two orthogonally oriented polarizers and each polarization image detected by an individual CCD camera in each path, as will be understood by a person having ordinary skill in the art.

Alternatively, the light emitted from the tissue sample may be spectrally filtered and passed through a polarization selection unit comprising a liquid crystal variable retarder and a linear polarizer that is oriented orthogonally to the illumination polarization. The liquid crystal variable retarder can be controlled to selectively rotate the polarization of the light emitted from the tissue sample prior to passing it through the linear polarizer, such that the fixed linear polarizer can act as a cross, 45 degree, parallel, or any other angle of polarization filter and the signal from each state can be sequentially captured with a single CCD camera.

In FIG. 7, an end cap 322 according to the present invention is adapted for use with the afore-described dermoscope system 300. It is shown in FIG. 7 coupled to the optics 302 of the system 300, which would be packaged as shown in FIGS. 1 and 2, described above.

An end-cap of the present invention might also be deployed in a multimode endoscopic measurement delivering hyperspectral light though a light pipe or optical fiber, and receiving emitted light through the same or a separate light pipe or optical fiber. Applicable polarization selection and spectral filtering methods may be selected by a person having ordinary skill in the art.

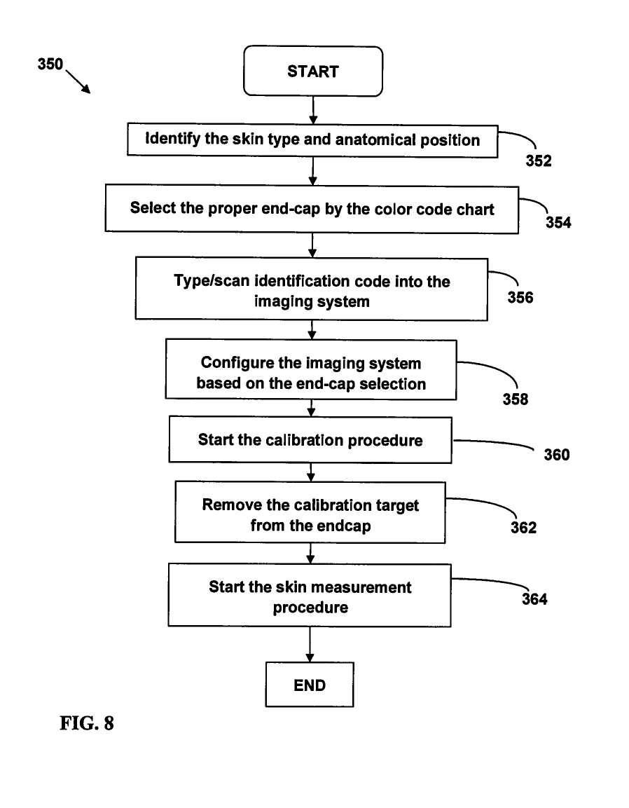

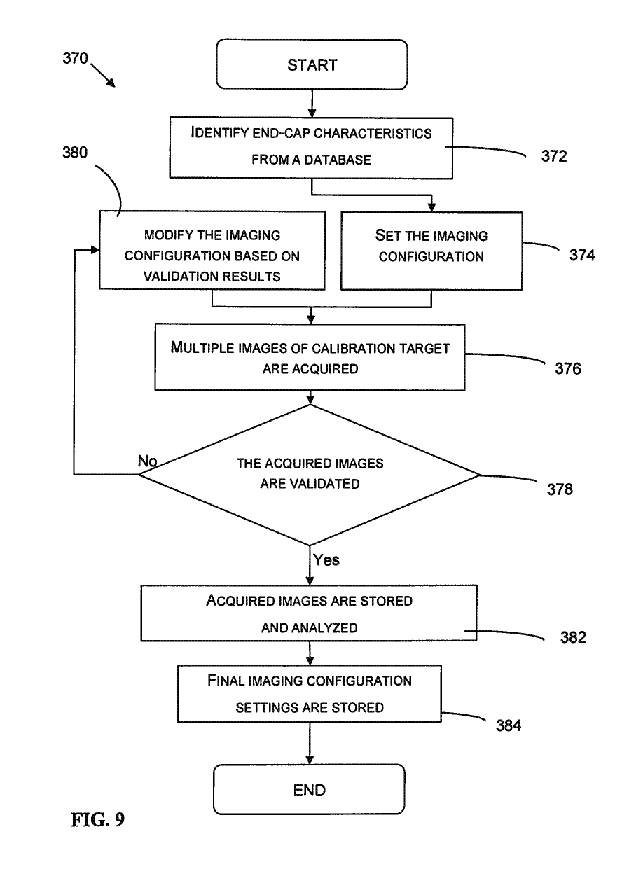

A dermoscope, such as one including a system 300 as described above, must be calibrated by scanning a reference target to correct illumination inhomogeneities, adjust exposure time for each spectral band to ensure an acceptable signal to noise ratio, subtract the dark current image, remove hot or bad pixel defects from the camera, and store the instrument spectral response characteristics so that the measured tissue optical data become independent of system responses and reflect the true characteristic response of the tissue. FIG. 8 shows an overview of typical steps (350) carried out to calibrate the dermoscope then initiate skin measurement. They may comprise identifying the skin type and anatomical position of measurement (352); selecting the end-cap with the proper skin type, size and shape characteristics by a color code chart (354); scanning the end cap unique identifier 114 into the imaging system (356); configuring the imaging acquisition system based on the end-cap characteristics (358); starting the calibration procedure (described hereafter) (360); removing the end cap after calibration is complete (362) to make the skin visible to the detection part of dermoscope hand-piece so that the skin may be examined with the probe; and starting the skin measurement (364).

A representative calibration method 370 according to the invention, implemented at step 360 of FIG. 8, is illustrated in FIG. 9. First the dermoscope system determines the end-cap characteristics from a database of known characteristics linked to the unique identifier (372); the dermoscope imaging acquisition system configuration is initialized, that is, the imaging configuration is set, based on the end-cap characteristics (374); multiple images of the calibration target are sequentially acquired at different wavelengths and at different polarization states (376); the acquired images are validated by comparing the measured image data to the expected data to make sure they are suitable for analysis (378); if necessary the image configuration setup is modified and images of the calibration target are re-acquired until they are suitable for analysis (380); otherwise the acquired images are stored and analyzed by comparing measured values to known values for the target (382); and, calibration correction factors are computed such that the corrected images correspond to the known and expected values for the calibration target and the final imaging configuration settings are stored (384).

The database of the known characteristics may include the shape of the end-cap, the lot number of the components used in the manufacture of the end-cap, the measured reference target response, whether the end-cap has been used before, and other characteristics that may be useful. The dermoscope imaging acquisition system configuration includes adjusting the exposure time, wavelength ranges, polarization settings, illumination power, camera gain, pixel binning, and other similar image acquisition settings. The image validation checks that the image has sufficient brightness for analysis, that the image is not saturated, that the image is not out of focus, that all the images in the image set are captured, that the images are not blurred due to unwanted dermoscope or target movement, that the images are captured within the allowable temperature range and other similar factors. The calibration process produces correction factors that correct for the wavelength dependent response and spatially dependent response for each pixel. These correction factors can be stored in a form of multi-dimensional image data cube.

A multimode multispectral/hyperspectral dermoscope such as the SkinSpect™ multimode imaging system provided by Spectral Molecular Imaging Inc., Beverly Hills, Calif., can combine hyper-spectral, polarization, and autofluorescence imaging modalities to capture images of the skin for analysis.

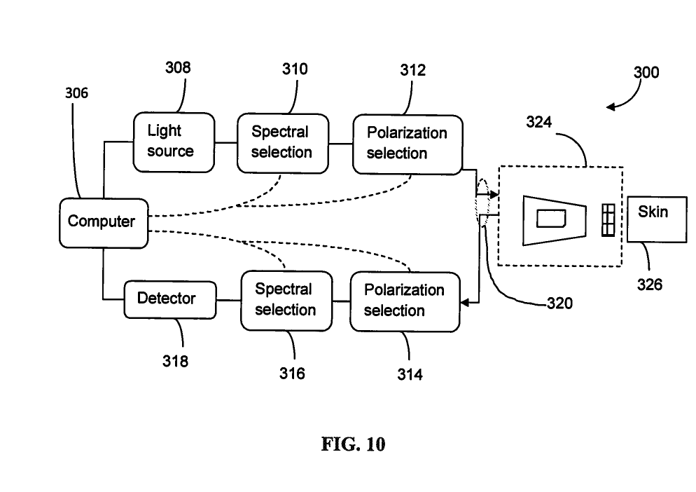

FIG. 10 illustrates the dermoscope system 300 described with respect to FIG. 8, but with the end cap configured for skin measurement as shown at 324, that is, with the calibration target removed. The end cap is then placed against the skin 326. A representative skin measurement method according to the invention then proceeds as follows.

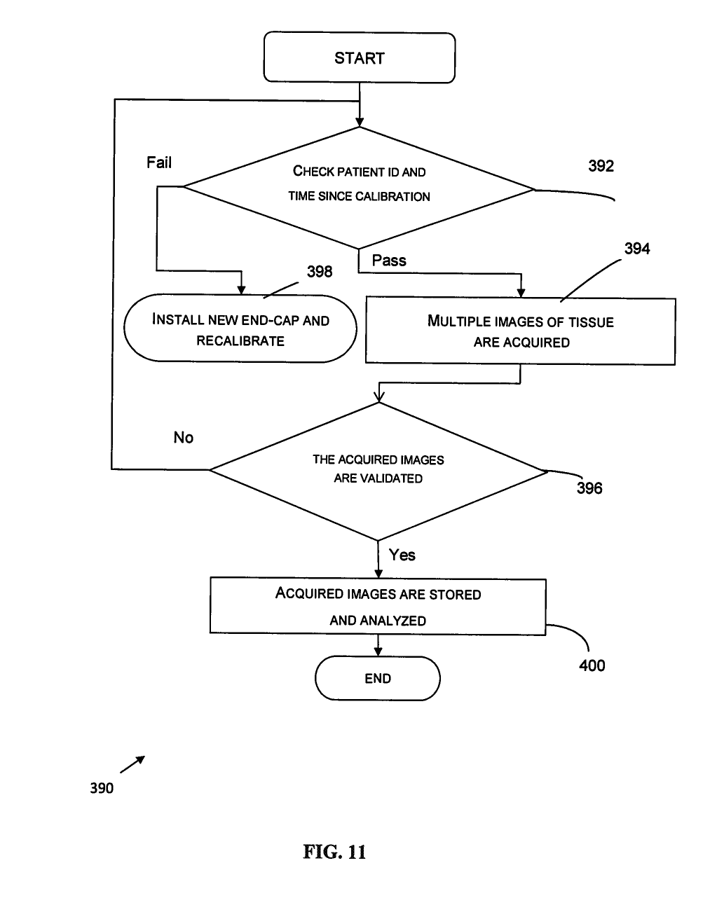

Referring to FIG. 11, in the method for skin measurement 390 the dermoscope system first checks to verify that the patient is the same for which the end cap was calibrated and to determine that the time since calibration is within allowable limits (392). In a preferred embodiment a reasonable period of time between the calibration and skin measurement is the time that the instrument will not have physical and environmental changes that significantly affect the imaging response, the length of that time depending, among other things on the physical characteristics of the particular device and environmental conditions a typical maximum allowable period of time would be in a range of 10-60 minutes, but preferably less than 30 minutes. If too much time has passed the calibration process should be performed again with a new end-cap. To proceed, the operator must make sure that the calibration target is removed from the end-cap and the aseptic surface is touching the patient skin for the measurement. Next, multiple images of the skin are sequentially acquired at different wavelengths and at different polarization states (394). The acquired images are validated by comparing the measured image data to the expected data to make sure they are suitable for analysis (396). If the images fail validation or are not appropriately located in the field of view, the images need to be re-acquired (398). Otherwise, the acquired images are analyzed and stored (400). The operator may optionally make additional measurements with the same subject if desired and within the allowed period of time following the calibration. To ensure adequate calibration and minimize possible unsanitary reuse of the end-cap the system software may be adapted to lock out further measurements until a new end-cap is installed.

In general, a multimode, multispectral or hyperspectral imaging system may be used with tissues other than the dermis of the skin. For example, the imaging system may be used to examine open wounds, or tissues exposed during the surgery. In such cases, calibration target with different optical properties, such as those corresponding to wounds like chronic ulcers, will be required to maintain optical system accuracy. It is a further object of this invention to provide calibration targets suitable for tissues other than skin. In addition, the end-cap may be used with a dermoscope applied to animals other than a human being.

The terms and expressions which have been employed in the foregoing specification are used therein as terms of description and not of limitation, and there is no intention, in the use of such terms and expressions, to exclude equivalents of the features shown and described or portions thereof, it being recognized that the scope of the invention is defined and limited by the claims that follow.

1. An end cap for use with a dermoscope having a scope aperture adapted to emit light to illuminate animal tissue and receive light emitted from the tissue in response to illumination of the tissue, and a data processor adapted to process data regarding light emitted from the tissue, comprising:
a tube having a first end forming a tube aperture adapted to receive light from and transmit light into the scope aperture, and a second end;
a calibration target adapted to be removably disposed at the second end of the tube so as to receive light from the dermoscope through the tube aperture and the scope aperture; and
an end cap identifier disposed on the end cap so as to uniquely identify the end cap so that the data processor may associate the end cap with data regarding light emitted from an individual animal and calibration data derived from light received from the calibration target.

2. The end cap of claim 1, wherein the calibration target has optical response characteristics corresponding to a particular skin type.

3. The end cap of claim 2, wherein the optical response characteristics include spectral response characteristics.

4. The end cap of claim 2, wherein the optical response characteristics include polarization response characteristics.

5. The end cap of claim 4, wherein the optical response characteristics include spectral response characteristics.

6. The end cap of claim 2, wherein the optical response characteristics include fluorescence response characteristics.

7. The end cap of claim 2, wherein the characteristics based on a particular skin type correspond to one of a plurality of standardized skin types having specific absorption and scattering characteristics.

8. The end cap of claim 2, wherein the end cap identifier includes data that identifies the optical response characteristics of the end cap.

9. The end cap of claim 1, wherein the tube is shaped to match and attach to the dermoscope over the scope aperture.

10. The end cap of claim 8, wherein the tube is larger at the first end than at the second end.

11. The end cap of claim 1, wherein, with the calibration target removed from the tube, the tube is opened at both the first end and the second end.

12. The end cap of claim 1, further including an elastomeric ring attached to the second end of the tube and disposed between the second end of the tube and the calibration target.

13. The end cap of claim 11, wherein the calibration target is removably attached to the elastomeric ring.

14. The end cap of claim 1, wherein the end cap identifier is a barcode.

15. The end cap of claim 1, wherein the end cap identifier is an RFID device.

16. The end cap of claim 1, wherein the end cap identifier is a two-dimensional symbol.

17. The end cap of claim 1, wherein the end cap identifier is removably attached to the end cap tube.

18. The end cap of claim 1, wherein the end cap is color coded to indicate the skin type with which it is to be used.

19. The end cap of claim 1, wherein the tube is shaped so as to be applied to a particular anatomical area of the animal with which it is to be used.

20. The end cap of claim 1, further comprising a computer program for use in a computer associated with the dermoscope and adapted to accept data from the end cap identifier and dermoscope measurements of the calibration target to calibrate the dermoscope for the skin type with which the dermoscope is to be used.

21. A method for calibrating a dermoscope having a scope aperture adapted to emit light to illuminate the animal tissue and receive light emitted from the animal tissue in response to illumination of the tissue, and a data processor adapted to process data regarding light emitted from the animal tissue, comprising:
identifying the skin type of an animal subject whose skin is to be examined;
selecting an end cap corresponding to the skin type of the animal subject, the end cap having a tube having a first end forming a tube aperture adapted to receive light from and transmit light into the scope aperture, and a second end, and a calibration target adapted to be removably disposed at the end of the tube so as to receive light from the dermoscope through the tube aperture and the scope aperture, the calibration target corresponding to the selected skin type;
entering data regarding the optical characteristics of the calibration target into the data processor; and
causing the data processor to calibrate the response of the dermoscope to take into account the assumed optical characteristics of the skin based on the optical characteristics of the calibration target.

22. The method of claim 21, wherein the optical characteristics of the calibration target to be considered include absorption and scattering.

23. The method of claim 22, wherein selecting the end cap is performed by reading an end cap identifier disposed on the end cap.

24. The method of claim 21, wherein the optical characteristics of the calibration target to be considered include fluorescence response characteristics.

25. The method of claim 22, wherein the end cap identifier is a bar code that includes information about optical characteristics of the calibration target and the bar code is read by a bar code scanner that provides information regarding the optical characteristics of the calibration target for input to the data processor.

26. The method of claim 23, wherein the end cap identifier is a two dimensional symbol that includes information about optical characteristics of the calibration target and the symbol is read by a scanner that provides information regarding the optical characteristics of the calibration target for input to the data processor.

27. The method of claim 23, wherein the end cap identifier is a RFID device that provides information about optical characteristics of the calibration target and is read by a RFID transceiver to obtain information regarding the optical characteristics of the calibration target for input to the data processor.

28. The method of claim 22, wherein selecting the end cap is performed by reading an end cap identifier disposed on the end cap.

29. The method of claim 28, wherein the end cap identifier is a bar code that includes information about optical characteristics of the calibration target and the bar code is read by a bar code scanner that provides information regarding the optical characteristics of the calibration target for input to the data processor.

30. The method of claim 28, wherein the end cap identifier is a two dimensional symbol that includes information about optical characteristics of the calibration target and the symbol is read by a scanner that provides information regarding the optical characteristics of the calibration target for input to the data processor.

31. The method of claim 28, wherein the end cap identifier is a RFID device that provides information about optical characteristics of the calibration target and is read by a RFID transceiver to obtain information regarding the optical characteristics of the calibration target for input to the data processor.

32. The method of claim 21, wherein the end cap identifier is a color code that includes information about optical characteristics of the calibration target for input to the data processor are determined by reading the color code.

33. The method of claim 21, further comprising causing the data processor to calibrate the response of the dermoscope to take into account the assumed optical characteristics of the skin based on the anatomical position at which the skin of the subject is to be examined.

34. A method for examining human/animal tissue to identify lesions comprising:
providing a dermoscope having a scope aperture adapted to emit light to illuminate a portion of the tissue and receive light emitted from the tissue in response to illumination of the tissue, and a data processor adapted to process data regarding light emitted from the animal tissue;
providing an end cap adapted to be placed on the scope aperture, the end cap having a calibration target and a unique identifier representing the type of end cap and optical characteristics of the calibration target;
reading the unique identifier to provide to the data processor the type of end cap and optical characteristics of the calibration target;
placing the end cap on the scope aperture;
taking one or more measurements of the calibration target;
calibrating the dermoscope based on the measurements of the calibration target, the type of calibration target and the optical characteristics of the calibration target;
removing the calibration target from the end cap;
illuminating the human/animal tissue with the calibration target over a plurality of wavelengths and a plurality of polarizations while measuring the reflected illumination light; and based on the preceding measurements of intensity, producing data representative of one or more characteristics of the tissue.
